(19) United States
(12) Patent Application Publication
(10) Pub. No.: US 2022/0409628 A1
(43) Pub. Date: Dec. 29, 2022

(54) 18-MC FOR TREATING OBESITY

(71) Applicant: Mind Medicine, Inc., New York, NY (US)

(72) Inventors: Robert BARROW, Madison, WI (US); Daniel R. KARLIN, New York, NY (US)

(73) Assignee: Mind Medicine, Inc., New York, NY (US)

(21) Appl. No.: 17/835,051

(22) Filed: Jun. 8, 2022

Related U.S. Application Data
(60) Provisional application No. 63/216,118, filed on Jun. 29, 2021.

(51) Int. Cl. A61K 31/55; A61P 3/04; A61P 25/30

(52) U.S. Cl. CPC A61K 31/55; A61P 3/04; A61P 25/30

(57) ABSTRACT

A method of treating obesity, by administering an effective amount of a composition chosen from the group consisting of 18-methoxycoronaridine (18-MC), salts thereof, tartrates thereof, solvates thereof, isomers thereof, analogs thereof, homologues thereof, and deuterated forms thereof to an individual and treating obesity. A method of treating binge eating, by administering an effective amount of the composition to an individual and treating binge eating. A method of treating behavioral addictions, by administering an effective amount of the composition to an individual and treating the behavioral addiction.

### BACKGROUND OF THE INVENTION

### 1. Technical Field

The present invention relates to compositions and methods for treating obesity and behavior addictions. More specifically, the present invention relates to methods of treating obesity with 18-methoxycoronaridine.

### 2. Background Art

Obesity is generally defined by the Centers for Disease Control and Prevention as having a body mass index of 30.0 or more based on a persons height and weight. More than 40 percent of American adults are considered obese. Causes of obesity can include genetics of how the body processes food and how fat is stored, growing older leading to less muscle mass and slower metabolic rates, lack of sleep that can cause hormonal changes and make one crave food, pregnancy, as well as other health conditions such as polycystic ovary syndrome, Prader-Willi syndrome, Cushing syndrome, hypothyroidism, or osteoarthritis.

Obesity leads to many other health complications such as type 2 diabetes, heart disease, cancers, stroke, gallbladder disease, fatty liver disease, high cholesterol, sleep apnea, arthritis, and infertility.

Treatments for obesity usually include lifestyle and behavioral changes, such as following a diet and increasing daily exercise. Some drugs can be used to prevent the absorption of fat or suppress the appetite, such as phentermine/topiramate, naltrexone/bupropion, liraglutide, or orlistat. Bariatric surgery is also used to treat obesity.

18-methoxycoronaridine (18-MC) is a derivative of ibogaine with the chemical formula of C₂₂H₂₈N₂O₃. 18-MC is an α₃β₄ nicotinic receptor antagonist. It has been used in reducing self-administration of morphine, cocaine, methamphetamine, nicotine, and sucrose (U.S. Pat. No. 6,780,871 to Glick, et al.).

Taraschenko, et al. (Psychopharmacology (Berl). 2008 December; 201(3): 339-350.) teach that acute administration of 18-MC (10-40 mg/kg i.p.) in rats reduced operant responding for sucrose and decreased ad libitum ingestion of sucrose, saccharin, and saline. The highest dose of 18-MC also reduced consumption of water when palatable fluids were not available. In rats having unlimited access to sucrose (30%), chronic treatment with 18-MC (20 mg/kg i.p.) prevented sucrose-induced increases in body weight, decreased fat deposition, and reduced consumption of sucrose while not altering food intake.

Taraschenko, et al. (Pharmacol Biochem Behav. 2010 September; 96(3): 247-250.) teach that 18-MC does not induce a conditioned taste aversion to sucrose.

McCallum, et al. (Psychopharmacology (Berl). 2011 May; 215(2): 247-256.) teach that pretreatment of female rats with 18-MC (20 mg/kg, i.p.), given prior to the administration of ghrelin (1 μg, lateral ventricle), blocked ghrelin-induced increases in sucrose (5%) intake in a two-bottle open access paradigm. Using in vivo microdialysis, 18-MC (both 20 and 40 mg/kg) prevented ghrelin (2 μg, intraventral tegmental area)-induced increases in extracellular dopamine in the nucleus accumbens. 18-MC had no effect on deposition of fat or on serum levels of glucose, triglycerides, and cholesterol in ghrelin-treated rats.

Taraschenko, et al. (Physiol Behav. 2011 Feb. 1; 102(2): 126-131.) teach that the effects of repeated administration of 18-MC on body weight gain, deposition of fat, consummatory behavior and biochemical markers of obesity in male rats were assessed. In contrast to females, males consuming ad libitum quantities of sucrose solution (30%) in combination with normal chow did not become obese; they did not gain excessive weight nor show excessive fat deposition. Repeated administration of 18- MC (20 mg/kg, i.p.) attenuated weight gain in both sucrose-consuming and control animals without altering food or fluid intake. The results indicate that males and females are differentially responsive to high carbohydrate-diet obesity. Such gender disparities could be secondary to sex-specific alterations in cholinergic mechanisms of feeding and body weight regulation.

While there have been experiments performed in rats showing that 18-MC has an effect on obesity, there is no suggestion that this would be the same in humans. There remains a need for an effective treatment for obesity and other behavioral addictions.

### SUMMARY OF THE INVENTION

The present invention provides for a method of treating obesity, by administering an effective amount of a composition chosen from the group consisting of 18-methoxycoronaridine (18-MC), salts thereof, tartrates thereof, solvates thereof, isomers thereof, analogs thereof, homologues thereof, and deuterated forms thereof to an individual and treating obesity.

The present invention also provides for a method of treating binge eating, by administering an effective amount of a composition chosen from the group consisting of 18-MC, salts thereof, tartrates thereof, solvates thereof, isomers thereof, analogs thereof, homologues thereof, and deuterated forms thereof to an individual and treating binge eating.

The present invention provides for a method of treating behavioral addictions, by administering an effective amount of a composition chosen from the group consisting of 18-MC, salts thereof, tartrates thereof, solvates thereof, isomers thereof, analogs thereof, homologues thereof, and deuterated forms thereof to an individual and treating the behavioral addiction.

### DETAILED DESCRIPTION OF THE INVENTION

The present invention provides for a method of treating obesity, by administering an effective amount of 18-MC to an individual and treating obesity.

The free base 18-methoxycoronaridine (18-MC) is a synthetic coronaridine congener and a specific negative allosteric modulator (antagonist) of α3β4 nicotinic cholinergic receptors; it indirectly modulates the dopaminergic mesolimbic pathway via blockade of α3β4 nicotinic receptors in the habenulo-interpeduncular pathway and the basolateral amygdala (Glick et al., 2008).

18-MC in the present invention can also be in the form of salts thereof, tartrates thereof, solvates thereof, isomers thereof, analogs thereof, homologues thereof, or deuterated forms thereof.

18-MC can be administered as a single dose or as repeat doses over multiple days, weeks, months, or years. 18-MC can be administered in a dose from 0.01-10 mg/kg.

18-MC can reduce craving for food, especially foods containing sucrose, and other related carbohydrates, thus reducing weight gain in the individual and obesity.

The present invention also provides for a method of treating binge eating, by administering an effective amount of 18-MC to an individual and treating binge eating. With binge eating disorder, an individual frequently consumes large amounts of food, and they feel unable to stop eating. A binge eating episode is defined as an individual eating in a discrete period of time (e.g., within any 2-hour period) an amount of food that is definitely larger than what most people would eat in a similar period of time under similar circumstances, and a sense of lack of control overeating during the episode (e.g., a feeling that one cannot stop eating or control what or how much one is eating). The binge eating episodes are associated with three (or more) of eating much more rapidly than normal, eating until feeling uncomfortably full, eating large amounts of food when not feeling physically hungry, eating alone because of feeling embarrassed by how much one is eating, or feeling disgusted with oneself, depressed, or very guilty afterward. A binge eater also does not regularly use unhealthy compensatory measures (e.g., purging) to counter the binge eating. 18-MC reduces the need to consume large amounts of food.

The present invention further provides for a method of treating behavioral addictions, by administering an effective amount of 18-MC to an individual and treating the behavioral addiction. Behavioral addictions are behaviors that often occur in response to a feeling of anxiety, urge, or negative feelings, and the behaviors can create a calming feeling in the individual and stimulate reward areas in the brain in a way that is understood to be similar to substances of misuse or abuse. The behavioral addiction can be, but is not limited to, gambling, sex, food, plastic surgery, social media, internet, risks, shopping, or pornography. 18-MC can reduce and/or eliminate the need to do the relevant behaviors when suffering from a behavioral addiction in the individual.

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants, and vehicles. The compounds can be administered orally, transcutaneously, subcutaneously or parenterally including sublingual, buccal, inhalation, intravenous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours, days, weeks, months, or years.

When administering the compound of the present invention orally, it will generally be formulated in an immediate release capsule, immediate release tablet, modified release capsule or tablet (including enteric coatings), solution, or suspension. When administering the compound of the present invention parenterally, it will generally be formulated in a sublingual or buccal orally dissolving tablet, dissolving film, intranasal powder, intranasal solution, inhaled powder, inhaled solution, transdermal patch, transdermal patch with microneedles or other permeation enhancers, or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of treating obesity, including the steps of:
administering an effective amount of a composition chosen from the group consisting of 18-methoxycoronaridine (18-MC), salts thereof, tartrates thereof, solvates thereof, isomers thereof, analogs thereof, homologues thereof, and deuterated forms thereof to an individual; and
treating obesity.

2. The method of claim 1, wherein the composition is administered in a dose of 0.01-10 mg/kg.

3. The method of claim 1, wherein the composition is administered as a single dose.

4. The method of claim 1, wherein the composition is administered as a repeat dose over a time period chosen from the group consisting of days, weeks, months, and years.

5. The method of claim 1, wherein said treating step further includes the steps of reducing craving for food and reducing weight gain in the individual.

6. A method of treating binge eating, including the steps of:
administering an effective amount of a composition chosen from the group consisting of 18-methoxycoronaridine (18-MC), salts thereof, tartrates thereof, solvates thereof, isomers thereof, analogs thereof, homologues thereof, and deuterated forms thereof to an individual; and
treating binge eating.

7. The method of claim 6, wherein the composition is administered in a dose of 0.01-10 mg/kg.

8. The method of claim 6, wherein the composition is administered as a single dose.

9. The method of claim 6, wherein the composition is administered as a repeat dose over a time period chosen from the group consisting of days, weeks, months, and years.

10. The method of claim 6, wherein said treating step further includes the step of reducing craving for food in the individual.

11. A method of treating behavioral addictions, including the steps of:
administering an effective amount of a composition chosen from the group consisting of 18-methoxycoronaridine (18-MC), salts thereof, tartrates thereof, solvates thereof, isomers thereof, analogs thereof, homologues thereof, and deuterated forms thereof to an individual; and
treating the behavioral addiction.

12. The method of claim 11, wherein the behavioral addiction is chosen from the group consisting of gambling, sex, food, plastic surgery, social media, internet, risks, shopping, and pornography.

13. The method of claim 11, wherein said treating step further includes the steps of reducing and/or eliminating a need to do behaviors relating to the behavioral addiction.

14. The method of claim 11, wherein the composition is administered in a dose of 0.01-10 mg/kg.

15. The method of claim 11, wherein the composition is administered as a single dose.

16. The method of claim 11, wherein the composition is administered as a repeat dose over a time period chosen from the group consisting of days, weeks, months, and years.
